(19) United States
(12) Patent Application Publication
(10) Pub. No.: US 2006/0069370 A1
(43) Pub. Date: Mar. 30, 2006

(54) Absorbent article having a liner with areas that prevent lotion and adhesive migration

(75) Inventors: Alissa Ellingson, Appleton, WI (US); Mike G. Shlepr, Greenville, WI (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 10/953,361

(22) Filed: Sep. 30, 2004

(51) Int. Cl. A61F 13/15

(52) U.S. Cl. 604/382

(57) ABSTRACT

An absorbent article is disclosed containing an absorbent structure positioned adjacent to a body-side liner. The body-side liner contains areas of relatively high permeability and areas of relatively low permeability. The relatively high permeability areas are for permitting rapid liquid transfer through the liner to the absorbent structure. The relatively low permeability areas, on the other hand, are for topical application of a chemical composition, such as a lotion composition or an adhesive composition to the liner. The relatively low permeability areas, for example, substantially inhibit the topically applied chemical composition from migrating through the liner to the opposite side. In this manner, a lotion composition applied to an exterior surface of the absorbent article is inhibited from being absorbed by the absorbent structure. An adhesive composition applied to the liner for attaching the liner to the absorbent structure, on the other hand, is prevented from migrating through the liner and irritating the wearer.

### BACKGROUND OF THE INVENTION

Disposable absorbent articles such as diapers, training pants, adult incontinence products, absorbent underpants, feminine care products, bandages, and nursing pads have been used to absorb body fluids and leave the skin dry. The absorbent articles typically include a liquid impermeable outer cover, an absorbent core, and a liquid permeable body-side liner. The absorbent core is typically located in between the outer cover and the liner for taking in and retaining fluids (e.g., urine) exuded by the wearer. In constructing the absorbent articles, the outer cover and the liner may be thermally or adhesively bonded together, while the absorbent core is adhesively secured to both the outer cover and the liner.

An unavoidable consequence of the use of absorbent articles is that the skin is exposed more directly to various physical and biological insults. In order to provide disposability, absorbent articles are primarily constructed of nonwoven materials. Even though nonwoven materials are engineered to have soft hand and drape, they rub against the skin and there is friction. Such friction constitutes one form of physical insult to the skin barrier.

In addition to the above physical insults, skin covered by absorbent articles is also frequently exposed to biological insults. Biological fluids, such as urine, feces, vaginal secretions and the like may contain a variety of components that can damage the skin barrier. Once the skin barrier is compromised, these components can initiate or exacerbate inflammation of the skin.

In the past, in order to protect the skin during use of absorbent articles, a lotion composition has been applied to the liner that can, for instance, transfer to the skin of the user. For example, various lotion compositions are disclosed in U.S. Pat. No. 6,756,520 to Krzysik, which is incorporated herein by reference.

Although the above patent has provided great advances in the art in protecting the skin during use of absorbent articles, further improvements are still needed. For example, body-side liners are, by requirement of function, of high enough porosity so as not to hinder fluid intake. The high permeability liners, however, present difficulties in deposition and retention of lotion compositions on the surface. In particular, the lotion compositions have a tendency to migrate through the liner and become absorbed by the absorbent core. Similarly, adhesives applied to the liner that are used to adhere the liner to the absorbent core also have a tendency to migrate through and become present on the inner surface of the absorbent article. Once present, the adhesives may create high friction areas that rub against the skin during use.

In view of the above, a need currently exists for a body-side liner to be incorporated into an absorbent article that is configured not only to efficiently permit the transfer of fluids from the body of the wearer to an absorbent core, but is also configured to enable lotion transfer to the skin while preventing adhesive from migrating through the liner.

### SUMMARY OF THE INVENTION

In general, the following disclosure is directed to absorbent articles having a uniquely configured body-side liner that is well suited to not only receiving a lotion composition for use in the absorbent article but is also well suited to receiving an adhesive for adhering the body-side liner to other components contained within the article.

For example, in one embodiment, the present invention is directed to an absorbent article comprising an outer cover, a body-side liner, and an absorbent structure positioned in between the outer cover and the body-side liner. The body-side liner includes a first side that is located adjacent to the absorbent structure and a second side that is configured to be placed next to a wearer of the absorbent article. In accordance with the present invention, the liner includes at least one area of relatively high permeability and at least one area of relatively low permeability. The area of high permeability is configured to allow liquids to flow therethrough for absorption by the absorbent structure.

A lotion composition is applied to the second side of the liner or the body-side of the liner. The lotion composition is located on the relatively low permeability area of the liner. The relatively low permeability area inhibits the lotion composition from migrating through the liner to the absorbent structure and allows for not only maximum contact between the lotion composition and the wearer but also allows for relatively thick lotion layers, which is especially beneficial when the lotion composition is intended to transfer to the user. In one particular embodiment, for example, the lotion composition is only applied to the liner where the relatively low permeability area is located.

In one embodiment, the liner may include a pattern of relatively low permeability areas and relatively high permeability areas. For instance, in one embodiment, the relatively low permeability areas may be arranged in columns. The columns, for instance, may extend from a front region of the absorbent article through a crotch region and to a back region. In one particular embodiment, the pattern of columns may be interrupted by an insult zone comprising a relatively high permeability area. The insult zone may be positioned generally in the crotch region and may extend into the front region.

In addition to columns, it should be understood, that any suitable pattern of relatively low permeability areas may be incorporated into the liner based upon the particular product and the desired result. For example, in other embodiments, the pattern may comprise discrete shapes, a grid, or diagonal rows.

In general, any suitable lotion composition may be applied to the liner in accordance with the present invention. The lotion composition, for instance, may be a cream or an ointment and may be provided on the article to prevent, for instance, diaper rash or any other skin ailment or act as a protective barrier to the skin. In one particular embodiment, the lotion composition can include a hydrophilic solvent, a high molecular weight polyethylene glycol, a fatty alcohol, an emulsifying surfactant, a natural fat, a natural oil, a sterol, a sterol derivative, an emollient, or mixtures thereof.

The body-side liner made in accordance with the present invention can be constructed using various methods and techniques. For example, in one embodiment, the body-side liner comprises a nonwoven material and the relatively low permeability areas are formed by applying a film-forming material to the nonwoven material. In one particular embodiment, the film-forming material may be water soluble such that after being insulted with a body fluid, the film-forming material dissolves allowing for fluids to flow therethrough to the absorbent structure. The film-forming material may be, for instance, polyvinyl alcohol, polyvinyl acetate, polyethylene oxide, polypropylene oxide, polyacrylic acid salts, polyacrylamide, cellulose ethers, modified collagen gelatins, alginates, and modified starches.

In an alternative embodiment, the body-side liner comprises a nonwoven material that contains synthetic fibers. The nonwoven material may be, for instance, a meltspun web, such as a spunbond web, a bonded carded web, an airlaid web, or the like.

In one embodiment, the nonwoven material may be apertured. The relatively low permeability areas are then thermoformed into the nonwoven material. For example, the relatively low permeability areas may be formed into the nonwoven material by using a heated embossing roll and/or a non-heated pressure bonding roll. During this process, the permeability of the nonwoven material is reduced by causing the synthetic fibers to melt and spread out. In one particular embodiment, for instance, a film is created during the thermoforming process.

In still another embodiment of the present invention, the body-side liner comprises a laminate. In this embodiment, for instance, pieces of material having a desired shape may be laminated to the body-side liner to form the relatively low permeability areas.

In still another embodiment of the present invention, the body-side liner comprises a nonwoven material. The relatively low permeability areas may be made from the same material as the remainder of the body-side liner. The body-side liner may be formed, however, such that the relatively low permeability areas have a higher basis weight than the remainder of the material.

As described above, in one embodiment, the relatively low permeability areas provide a location for the placement of a lotion composition so that the lotion composition will not migrate through to the absorbent structure. In another embodiment, however, the relatively low permeability areas may be used to receive an adhesive that adheres the liner to the absorbent structure. In this manner, the adhesive is inhibited from migrating through the liner to the side of the liner that contacts the wearer. The adhesive may be, for instance, a hotmelt adhesive or a spray adhesive and may be applied to the liner only in the places where the relatively low permeability areas are located. Examples of adhesives that may be used include, for instance, a styrene-butadiene copolymer, a styrene-butadiene-styrene copolymer, a styrene-isoprene copolymer, an aliphatic polyolefin copolymer, an ethylene vinyl acetate copolymer, or a starch-based adhesive.

In still another embodiment of the present invention, the absorbent article may be constructed such that a lotion composition is applied to the relatively low permeability areas of the liner on one side and an adhesive is applied to the relatively low permeability areas on the opposite side of the liner. In this embodiment, the relatively low permeability areas perform a dual function of receiving a lotion composition and receiving an adhesive without either material migrating through the material.

Other aspects and features of the present invention are discussed in greater detail below.

### BRIEF DESCRIPTION OF THE DRAWINGS

A full and enabling disclosure of the present invention, including the best mode thereof to one of ordinary skill in the art, is set forth more particularly in the remainder of the specification, including reference to the accompanying figures, in which:

FIG. 1 is a rear perspective view of one embodiment of an absorbent article made in accordance with the present invention;

FIG. 2 is a front perspective view of the absorbent article illustrated in FIG. 1;

FIG. 3 is a plan view of the absorbent article shown in FIG. 1 with the article in an unfastened, unfolded and laid flat condition showing the surface of the article that faces away from the wearer;

FIG. 4 is a plan view similar to FIG. 3 showing one embodiment of the surface of the absorbent article that faces the wearer when worn;

FIG. 5 is a plan view similar to FIG. 3 showing another embodiment of the surface of the absorbent article that faces the wearer when worn;

FIG. 6 is a plan view similar to FIG. 3 showing another embodiment of the surface of the absorbent article that faces the wearer when worn;

FIG. 7 is a plan view similar to FIG. 3 showing another embodiment of the surface of the absorbent article that faces the wearer when worn;

FIG. 8 is a perspective view of one embodiment of a process for forming a body-side liner in accordance with the present invention;

FIG. 9 is a perspective view of another embodiment of a process for forming a body-side liner in accordance with the present invention; and

FIG. 10 is a plan view of another embodiment of a surface of an absorbent article that is intended to face the wearer when worn.

Repeated use of reference characters in the present specification and drawings is intended to represent the same or analogous features or elements of the present invention.

### DETAILED DESCRIPTION OF THE INVENTION

It is to be understood by one of ordinary skill in the art that the present discussion is a description of exemplary embodiments only, and is not intended as limiting the broader aspects of the present invention.

In general, the present invention is directed to absorbent articles that contain a body-side liner that not only allows liquids to pass through the liner to an absorbent structure, but are also well suited to receiving chemical compositions that are intended to remain on the surface of the liner. The chemical composition may be, for instance, a lotion composition that is intended to provide various wellness benefits to the user or, alternatively, an adhesive composition that is intended to attach the body-side liner to an adjacent surface. In order to accomplish the above two objectives, body-side liners made in accordance with the present invention include at least one area of relatively high permeability for fluid transfer and at least one area of relatively low permeability. In one embodiment, the body-side liner includes a pattern of relatively high permeability areas and relatively low permeability areas.

The purpose of the relatively low permeability areas is to provide a planar surface for topical application of a chemical composition, such as a lotion composition or an adhesive composition. As used herein, the low permeability areas are in reference to the chemical composition that is applied to the liner. For example, when applying a lotion composition that is hydrocarbon based, the relatively low permeability areas have a relatively low permeability area to hydrocarbon-based compositions. Likewise, when applying an adhesive composition to the liner, the relatively low permeability areas have a relatively low permeability to the adhesive composition. The relatively high permeability areas, on the other hand, have a relatively high permeability with respect to the fluid that is intended to pass through the liner. The fluid may be, for instance, urine or, in other embodiments, a menstrual fluid.

The relatively low permeability areas provide various benefits and advantages when incorporated into a liner in accordance with the present invention. When applying a lotion composition, for instance, the relatively low permeability areas facilitate formation of a thicker film of lotion, which is especially beneficial when the lotion composition is intended to transfer to the skin of a user. For instance, the relatively low permeability areas are constructed with sufficient surface area and low porosity that movement of the lotion composition, both during and after application, through the body-side liner to an absorbent structure is significantly inhibited. In all, the lotion composition remains in the position of optimal transfer to a user.

In addition to receiving a lotion composition, the relatively low permeability areas also provide for a contact area with an adhesive that is applied to the back side of the liner for attaching the liner to a subsequent layer, such as an absorbent structure. Again, the relatively low permeability areas prevent the adhesive composition from migrating through the liner to the surface of the liner that is to contact a user. The relatively low permeability areas are constructed with sufficient surface area to also prevent against blousing of the liner. The relatively high permeability areas, however, are also present in sufficient area for optimized fluid intake.

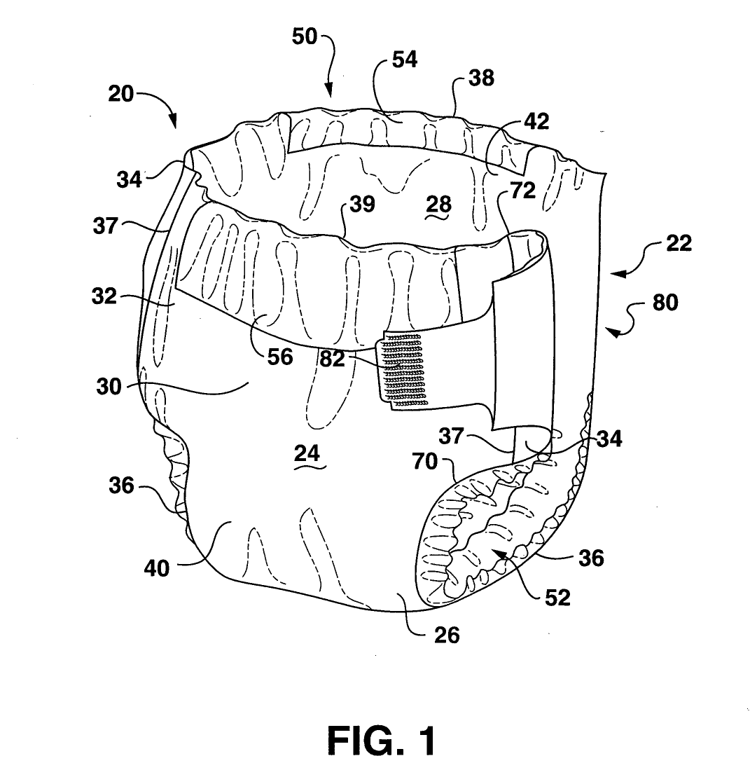

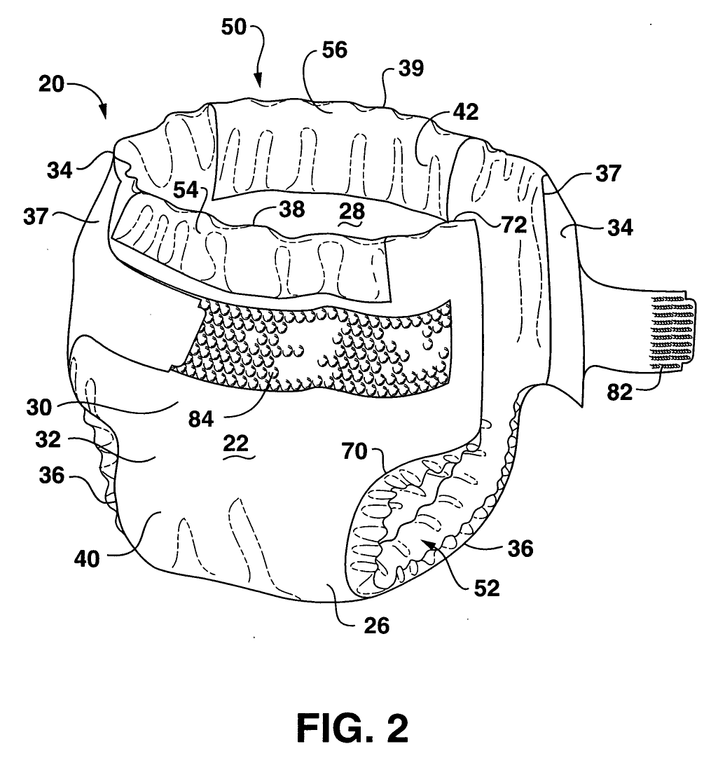

Referring to FIGS. 1 and 2, for exemplary purposes, an absorbent article 20 that may be made in accordance with the present invention is shown. The absorbent article 20 may or may not be disposable, which refers to articles that are intended to be discarded after a limited period of use instead of being laundered or otherwise conditioned for reuse. It is understood that the present invention is suitable for use with various other absorbent articles intended for personal wear, including but not limited to diapers, feminine hygiene products, incontinence products, medical garments, surgical pads and bandages, other personal care or health care garments, and the like without departing from the scope of the present invention.

By way of illustration only, various materials and methods for constructing absorbent articles such as the diaper 20 of the various aspects of the present invention are disclosed in PCT Patent Application WO 00/37009 published Jun. 29, 2000 by A. Fletcher et al; U.S. Pat. No. 4,940,464 issued Jul. 10, 1990 to Van Gompel et al.; U.S. Pat. No. 5,766,389 issued Jun. 16, 1998 to Brandon et al., and U.S. Pat. No. 6,645,190 issued Nov. 11, 2003 to Olson et al. which are incorporated herein by reference to the extent they are consistent (i.e., not in conflict) herewith.

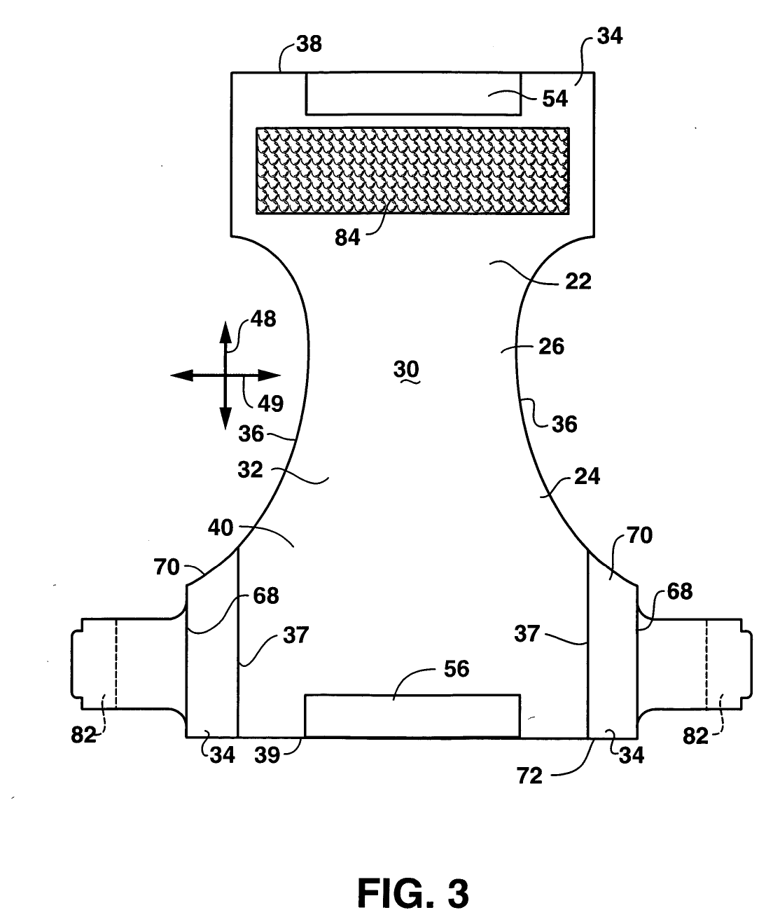

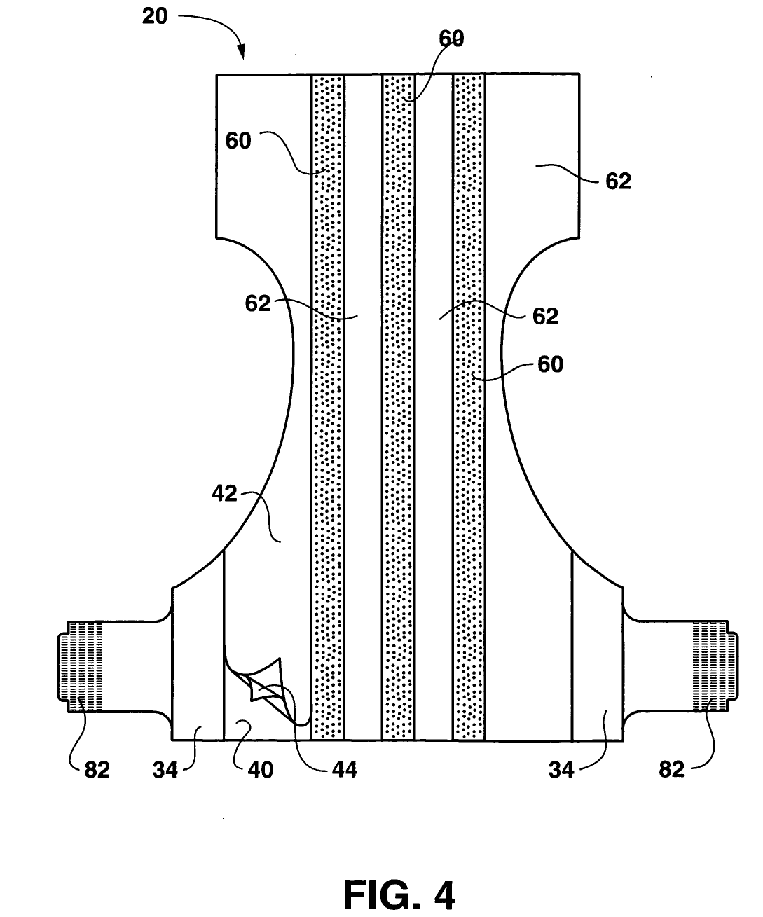

A diaper 20 is representatively illustrated in FIG. 1 in a partially fastened condition. The diaper 20 shown in FIGS. 1 and 2 is also represented in FIGS. 3 and 4 in an opened and unfolded state. Specifically, FIG. 3 is a plan view illustrating the exterior side of the diaper 20, while FIG. 4 illustrates the interior side of the diaper 20. As shown in FIGS. 3 and 4, the diaper 20 defines a longitudinal direction 48 that extends from the front of the article when worn to the back of the article. Opposite to the longitudinal direction 48 is a lateral direction 49.

The diaper 20 defines a pair of longitudinal end regions, otherwise referred to herein as a front region 22 and a back region 24, and a center region, otherwise referred to herein as a crotch region 26, extending longitudinally between and interconnecting the front and back regions 22, 24. The diaper 20 also defines an inner surface 28 adapted in use (e.g., positioned relative to the other components of the article 20) to be disposed toward the wearer, and an outer surface 30 opposite the inner surface. The front and back regions 22, 24 are those portions of the diaper 20, which when worn, wholly or partially cover or encircle the waist or mid-lower torso of the wearer. The crotch region 26 generally is that portion of the diaper 20 which, when worn, is positioned between the legs of the wearer and covers the lower torso and crotch of the wearer. The absorbent article 20 has a pair of laterally opposite side edges 36 and a pair of longitudinally opposite waist edges, respectively designated front waist edge 38 and back waist edge 39.

The illustrated diaper 20 includes a chassis 32, that, in this embodiment, encompasses the front region 22, the back region 24, and the crotch region 26. Referring to FIGS. 1-4, the chassis 32 includes an outer cover 40 and a bodyside liner 42 (FIGS. 1 and 4) that may be joined to the outer cover 40 in a superimposed relation therewith by adhesives, ultrasonic bonds, thermal bonds or other conventional techniques. The liner 42, for instance, may suitably be joined to the outer cover 40 along the perimeter of the chassis 32 to form a front waist seam, a back waist seam and a pair of side seams. The liner 42 can be generally adapted, i.e., positioned relative to the other components of the article 20, to be disposed toward the wearer's skin during wear of the absorbent article. The chassis 32 may further include an absorbent structure 44 particularly shown in FIG. 4 disposed between the outer cover 40 and the bodyside liner 42 for absorbing liquid body exudates exuded by the wearer, and may further include a pair of containment flaps (not shown) secured to the bodyside liner 42 for inhibiting the lateral flow of body exudates.

The elasticized containment flaps, for example, may define a partially unattached edge which assumes an upright configuration in at least the crotch region of the diaper to form a seal against the wearer's body. The containment flaps can extend longitudinally along the entire length of the chassis 32 or may extend only partially along the length of the chassis. Suitable constructions and arrangements for the containment flaps are generally well known to those skilled in the art and are described in U.S. Pat. No. 4,704,116 issued Nov. 3, 1987 to Enloe, which is incorporated herein by reference.

To further enhance containment and/or absorption of body exudates, the diaper 20 may also suitably include leg elastic members, as are known to those skilled in the art. The leg elastic members can be operatively joined to the outer cover 40 and/or the bodyside liner 42 and positioned in the crotch region 26 of the absorbent article 20.

The leg elastic members can be formed of any suitable elastic material. As is well known to those skilled in the art, suitable elastic materials include sheets, strands or ribbons of natural rubber, synthetic rubber, or thermoplastic elastomeric polymers. The elastic materials can be stretched and adhered to a substrate, adhered to a gathered substrate, or adhered to a substrate and then elasticized or shrunk, for example with the application of heat, such that elastic retractive forces are imparted to the substrate. In one particular aspect, for example, the leg elastic members may include a plurality of dry-spun coalesced multifilament spandex elastomeric threads sold under the trade name LYCRA and available from Invista, Wilmington, Del., U.S.A.

In some embodiments, the absorbent article 20 may further include a surge management layer (not shown) which may be optionally located adjacent the absorbent structure 44 and attached to various components in the article 20 such as the absorbent structure 44 or the bodyside liner 42. A surge management layer helps to decelerate and diffuse surges or gushes of liquid that may be rapidly introduced into the absorbent structure of the article. Desirably, the surge management layer can rapidly accept and temporarily hold the liquid prior to releasing the liquid into the storage or retention portions of the absorbent structure. Examples of suitable surge management layers are described in U.S. Pat. No. 5,486,166; and U.S. Pat. No. 5,490,846. Other suitable surge management materials are described in U.S. Pat. No. 5,820,973. The entire disclosures of these patents are hereby incorporated by reference herein to the extent they are consistent (i.e., not in conflict) herewith.

As shown in FIGS. 1-4, the absorbent article 20 further includes a pair of opposing elastic side panels 34 that are attached to the back region of the chassis 32. As shown particularly in FIGS. 1 and 2, the side panels 34 may be stretched around the waist and/or hips of a wearer in order to secure the garment in place. As shown in FIGS. 3 and 4, the elastic side panels are attached to the chassis along a pair of opposing longitudinal edges 37. The side panels 34 may be attached or bonded to the chassis 32 using any suitable bonding technique. For instance, the side panels 34 may be joined to the chassis by adhesives, ultrasonic bonds, thermal bonds, or other conventional techniques.

In an alternative embodiment, the elastic side panels may also be integrally formed with the chassis 32. For instance, the side panels 34 may comprise an extension of the bodyside liner 42, of the outer cover 40, or of both the bodyside liner 42 and the outer cover 40.

In the embodiments shown in the figures, the side panels 34 are connected to the back region of the absorbent article 20 and extend over the front region of the article when securing the article in place on a user. It should be understood, however, that the side panels 34 may alternatively be connected to the front region of the article 20 and extend over the back region when the article is donned.

With the absorbent article 20 in the fastened position as partially illustrated in FIGS. 1 and 2, the elastic side panels 34 may be connected by a fastening system 80 to define a 3-dimensional diaper configuration having a waist opening 50 and a pair of leg openings 52. The waist opening 50 of the article 20 is defined by the waist edges 38 and 39 which encircle the waist of the wearer.

In the embodiments shown in the figures, the side panels are releasably attachable to the front region 22 of the article 20 by the fastening system. It should be understood, however, that in other embodiments the side panels may be permanently joined to the chassis 32 at each end.

The elastic side panels 34 each have a longitudinal outer edge 68, a leg end edge 70 disposed toward the longitudinal center of the diaper 20, and waist end edges 72 disposed toward a longitudinal end of the absorbent article. The leg end edges 70 of the absorbent article 20 may be suitably curved and/or angled relative to the lateral direction 49 to provide a better fit around the wearer's legs. However, it is understood that only one of the leg end edges 70 may be curved or angled, such as the leg end edge of the back region 24, or alternatively, neither of the leg end edges may be curved or angled, without departing from the scope of the present invention. The outer edges 68 are generally parallel to the longitudinal direction 48 while the waist end edges 72 are generally parallel to the transverse axis 49. It should be understood, however, that in other embodiments the outer edges 68 and/or the waist edges 72 may be slanted or curved as desired. Ultimately, the side panels 34 are generally aligned with a waist region of the chassis.

The fastening system 80 may include laterally opposite first fastening components 82 adapted for refastenable engagement to corresponding second fastening components 84. In the embodiment shown in the figures, the first fastening component 82 is located on the elastic side panels 34, while the second fastening component 84 is located on the front region 22 of the chassis 32. In one aspect, a front or outer surface of each of the fastening components 82, 84 includes a plurality of engaging elements. The engaging elements of the first fastening components 82 are adapted to repeatedly engage and disengage corresponding engaging elements of the second fastening components 84 to releasably secure the article 20 in its three-dimensional configuration.

The fastening components 82, 84 may be any refastenable fasteners suitable for absorbent articles, such as adhesive fasteners, cohesive fasteners, mechanical fasteners, or the like. In particular aspects the fastening components include mechanical fastening elements for improved performance. Suitable mechanical fastening elements can be provided by interlocking geometric shaped materials, such as hooks, loops, bulbs, mushrooms, arrowheads, balls on stems, male and female mating components, buckles, snaps, or the like.

In the illustrated aspect, the first fastening components 82 include hook fasteners and the second fastening components 84 include complementary loop fasteners. Alternatively, the first fastening components 82 may include loop fasteners and the second fastening components 84 may be complementary hook fasteners. In another aspect, the fastening components 82, 84 can be interlocking similar surface fasteners, or adhesive and cohesive fastening elements such as an adhesive fastener and an adhesive-receptive landing zone or material; or the like. One skilled in the art will recognize that the shape, density and polymer composition of the hooks and loops may be selected to obtain the desired level of engagement between the fastening components 82, 84. Suitable fastening systems are also disclosed in the previously incorporated PCT Patent Application WO 00/37009 published Jun. 29, 2000 by A. Fletcher et al. and the previously incorporated U.S. Pat. No. 6,645,190 issued Nov. 11, 2003 to Olson et al.

In the embodiment shown in the figures, the fastening components 82 are attached to the side panels 34 along the edges 68. In this embodiment, the fastening components 82 are not elastic or extendable. In other embodiments, however, the fastening components may be integral with the side panels 34. For example, the fastening components may be directly attached to the side panels 34 on a surface thereof.

In addition to the side panels 34, the absorbent article 20 may also include various other elastic components. For example, as shown in FIGS. 1-3, the absorbent article 20 may further include a front waist elastic member 54 and a rear waist elastic member 56. The waist elastic members 54 and 56 may be added in order to control the stretch properties of the waist region.

As described above, the present invention is particularly directed to a body-side liner that contains relatively high permeability areas for liquid transfer and relatively low permeability areas that provide a support surface for various chemical compositions, such as a lotion composition or an adhesive composition. Referring to FIG. 4, for instance, the liner 42 includes a plurality of relatively low permeability areas 60 separated by relatively high permeability areas 62. In this particular embodiment, for instance, the relatively low permeability areas 60 comprise a plurality of columns that extend from the front region of the absorbent article 20 to the back region.

The permeability of the relatively high permeability areas 62 and the permeability of the relatively low permeability areas 60 may vary depending upon the particular application. In general, the relatively high permeability areas 62 should be capable of efficiently allowing liquids to pass through the material and become absorbed by the absorbent structure 44.

The relatively low permeability areas, on the other hand, should be constructed so as to substantially prevent a chemical composition applied to the areas from migrating through to the other side. The particular chemical composition applied to the liner has an impact upon the permeability of the relatively low permeability areas. In one particular embodiment, the relatively low permeability areas 60 are liquid impervious. For example, in one embodiment, the relatively low permeability areas 60 comprise a film.

The body-side liner 42 as shown in FIG. 4 may be constructed in various ways in order to form the relatively low permeability areas 60 and the relatively high permeability areas 62. As described above, the relatively low permeability areas are constructed so as to preclude or decrease migration of the chemical composition applied to the liner, such as a lotion composition, an adhesive composition, or both. In general, the body-side liner 42 may be manufactured from a wide selection of web materials, such as porous foams, reticulated foams, apertured plastic films, woven and non-woven webs, or a combination of any such materials.

In one particular embodiment, the body-side liner 42 comprises a web containing a heat fusible material. For example, the body-side liner 42 may comprise a non-woven material containing synthetic fibers or containing a binding agent. In one particular embodiment, for example, the body-side liner 42 may be a meltspun web, such as a meltblown web or a spunbond web.

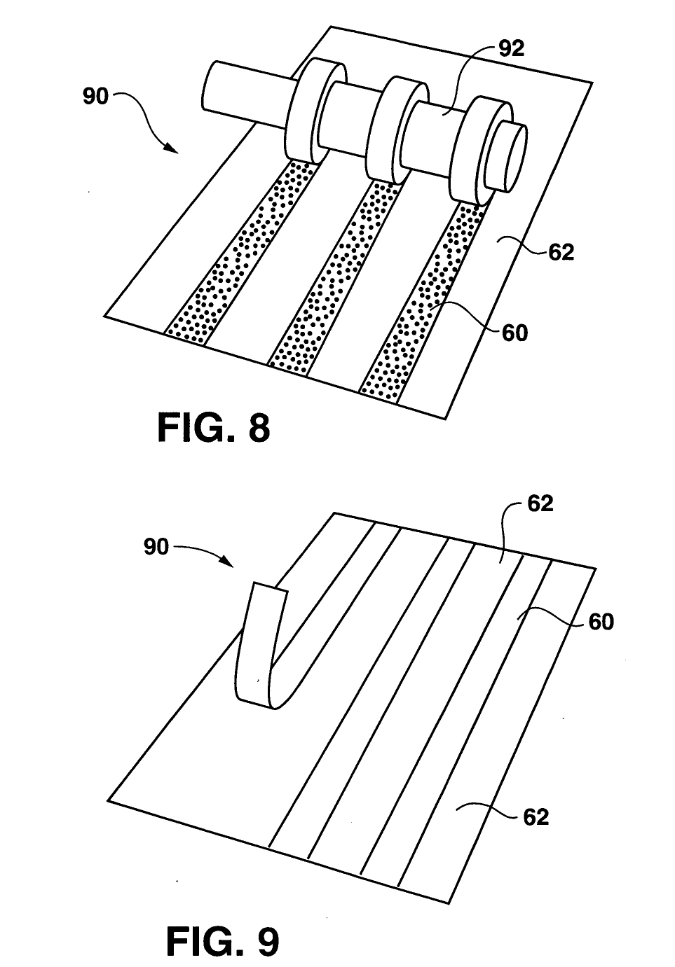

When the body-side liner 42 contains a heat fusible material, the relatively low permeability areas 60 may be thermoformed into the material. For example, referring to FIG. 8, one embodiment of a process for forming the relatively low permeability areas 60 is shown. As illustrated, in this embodiment, a liner material 90 is embossed by an embossing roll 92 in order to form the relatively low permeability areas 60. When fed through the embossing roll 92, the heat fusible material contained within the liner sheet 90 fuses together, thereby reducing the permeability of the sheet within the areas 60.

The embossing roll 92 may form the relatively low permeability areas 60 in various ways. For example, in one embodiment, the embossing roll 92 may be heated for thermally bonding the sheet 90 in order to form the relatively low permeability areas 60. In an alternative embodiment, the embossing roll may ultrasonically form the areas 60. In still another embodiment, pressure bonding is used in order to form the areas 60. In this embodiment, the relatively low permeability areas 60 may be slightly porous or may be formed into a film during the process and be completely liquid impervious.

Referring to FIG. 9, an alternative embodiment for forming the relatively low permeability areas 60 is shown. In this embodiment, the relatively low permeability areas 60 are formed by laminating strips of material to the body-side liner material 90. The strips may be adhesively secured to the liner material 90 or may be thermally bonded to the liner 90. The strips of material may be formed from the same material as the liner 90 or from any other suitable material.

In still another embodiment of the present invention, the relatively low permeability areas 60 may be formed by applying a film-forming composition to a liner material. The film-forming composition may be, for instance, a polyvinyl alcohol, a polyvinyl acetate, polyethylene oxide, polypropylene oxide, polyacrylic acid salts, polyacrylamide, cellulose ethers such as methyl cellulose, modified collagen gelatins, alginates such as agars, and modified starches. In one particular embodiment, the film-forming composition is water soluble so that the composition dissolves when the absorbent article is insulted with a body fluid. In this manner, once the absorbent article is insulted with a fluid, the relatively low permeability areas 60 dissolve allowing for a greater liquid transfer into the absorbent structure 44.

In other embodiments, however, a film-forming composition may be used that is not water soluble. In this embodiment, for example, the film-forming composition may comprise a polymer, such as a polyolefin. Particular polymers that may be used include polyethylene, polypropylene, nylons, polyesters, polystyrenes, and the like. In still other embodiments, the water insoluble film-forming composition may comprise a lotion composition or an adhesive composition that has been optimized for film forming. For example, the lotion composition may contain structurants, such as waxes that form a base for applying greater amounts of lotion to the liner material.

In one particular embodiment, for instance, a polyvinyl alcohol or a polyvinyl acetate is used as the film-forming composition. The polyvinyl alcohol or the polyvinyl acetate may be hydrolysed to an extent that renders the composition water soluble.

In still another embodiment of the present invention, the body-side liner 42 is formed from a molded non-woven web that includes high basis weight areas and low basis weight areas. The high basis weight areas which are formed into the web comprise the relatively low permeability areas, while the low basis weight areas comprise the relatively high permeability areas. In order to form the high and low basis weight areas, for instance, the non-woven webs may be formed on a forming surface under vacuum that includes a pattern of blocked off areas where the low basis weight areas are formed. Such webs may be formed, for instance, during a meltblown process, a spunbond process, an airlaid process, or a wetlay process.

In an alternative embodiment of the present invention, the body-side liner 42 comprises a low permeability material, such as a film or a nonwoven web. In this embodiment, the relatively high permeability areas are formed by perforating the material. The relatively low permeability areas, on the other hand, comprise areas on the material where the material has not been perforated. The perforations can be applied to the material in a pattern so that the relatively high permeability areas and the relatively low permeability areas form patterns on the material.

It should be understood that any of the above described techniques for forming relatively high permeability areas and relatively low permeability areas may be combined to form still another embodiment. For example, in one embodiment, a nonwoven may be embossed using heat and/or pressure to form the relatively low permeability areas while simultaneously being apertured at other locations for forming the relatively high permeability areas.

Once the liner 42 is formed containing the relatively high permeability areas 62 and the relatively low permeability areas 60, the liner can be incorporated into an absorbent article and the relatively low permeability areas may serve as a location for applying a chemical composition to the material. For example, in one embodiment, a lotion composition may be applied to the liner 42 on the side of the liner that faces the user. Due to the presence of the relatively low permeability areas 60, the lotion composition once applied to the liner is inhibited from migrating through the liner to the absorbent structure. Due to the relatively low permeability areas, a thicker film of the lotion composition may be formed on the article. Further, the lotion has a structure and is in position for optimal transfer to the user.

In general, any suitable lotion composition may be applied to the body-side liner 42 in accordance with the present invention. The lotion composition may be, for instance, an emulsion, a cream, an ointment, a salve, a suspension, encapsulations, a gel, and the like. The lotion composition can be applied to the liner using a variety of techniques including foam application, spraying, slot coating, and printing.

In one particular embodiment, the lotion composition can include a hydrophilic solvent, a high molecular weight polyethylene glycol, a fatty alcohol, an emulsifying surfactant, a natural fat, a natural oil, a sterol, a sterol derivative, an emollient, or mixtures thereof.

For example, the compositions can include from about 10 to about 90 percent by weight of one or more hydrophilic solvents. More specifically, the compositions can include from about 25 to about 75 percent by weight of hydrophilic solvents. Desirably, the compositions can include from about 30 to about 60 percent by weight of hydrophilic solvents. Hydrophilic solvents include, but are not limited to, water, propylene glycol, low molecular weight polyethylene glycols (molecular weights of less than 720 daltons and liquid at room temperature), methoxyisopropanol, PPG-2 propyl ether, PPG-2 butyl ether, PPG-2 methyl ether, PPG-3 methyl ether, dipropylene glycol propyl ether, dipropylene glycol butyl ether, dipropylene glycol, methyl propanediol, propylene carbonate, water soluble/dispersible polypropylene glycols, ethoxylated polypropylene glycol, glycerin, sorbitol solutions, hydrogenated starch hydrolysate, silicone glycols and mixtures of such compounds.

The compositions may also include from about 5 to about 95 percent by weight of one or more high molecular weight polyethylene glycols having a molecular weight of at least about 720 daltons. More specifically, the compositions can include from about 10 to about 50 percent by weight of high molecular weight polyethylene glycols. Desirably, the compositions can include from about 15 to about 25 percent by weight of high molecular weight polyethylene glycols. The high molecular weight polyethylene glycols primarily function to provide the hydrophilic solvents and any active ingredients in solid form. In addition to providing a solid medium for the solvent, and reducing its tendency to migrate, the high molecular weight polyethylene glycols provide a tackiness to the hydrophilic composition that improves transfer to the skin of the wearer. As used herein, suitable high molecular weight polyethylene glycols include, but are not limited to, the following materials: polyethylene glycols having an average molecular weight of 720 daltons or greater, and the like, as well as mixtures thereof. These materials are not liquid at room temperature. Particularly suitable high molecular weight polyethylene glycols can have an average molecular weight of from 720 to about 1,840,000 daltons, more specifically from about 1400 to about 440,000 daltons, and still more specifically from about 1760 to about 10,570 daltons.

The compositions may also include from about 1 to about 30 percent by weight of one or more fatty alcohols. More specifically, the compositions can include from about 10 to about 25 percent by weight of fatty alcohols. Desirably, the compositions of the invention can include from about 15 to about 20 percent by weight of fatty alcohols. As used herein, suitable fatty alcohols include, but are not limited to, the following materials: alcohols having a carbon chain length of C.sub.14-C.sub.30 or greater, including cetyl alcohol, stearyl alcohol, arachidyl alcohol, behenyl alcohol, and mixtures thereof.

The compositions may also include from about 1 to about 20 percent by weight of one or more emulsifying surfactants having an HLB range greater than 7 or a combination of low and high HLB surfactants that provide an HLB range greater than 7. More specifically, the compositions can include from about 2 to about 15 percent by weight of surfactants. Desirably, the compositions of the invention can include from about 3 to about 10 percent by weight of surfactants. Emulsifying surfactants are employed typically in cosmetic preparations to form emulsions of various components. The immiscible phase, such as an oil, is dispersed as droplets in the continuous phase, such as water or in this case the hydrophilic solvent. Suitable surfactants include, but are not limited to, Emulsifying Wax NF, Glyceryl Stearate, Glyceryl Stearate SE, Glycol Stearate, Glycol Stearate SE, Glycereth-20 Stearate, Glyceryl Behenate, Glyceryl Hydroxystearate, Glyceryl Laurate SE, Glyceryl Oleate, Glyceryl Oleate SE, Propylene Glycol Oleate, Propylene Glycol Oleate SE, Propylene Glycol Stearate, Propylene Glycol Stearate SE, Sorbitan Stearate, Sorbitan Trioleate, water dispersible metal soaps (Sodium Stearate), Behenyl Dimethicone Copolymers, Lauryl Methicone Copolymers, Cetyl Methicone Compolymers, Cetyl Dimethicone Copolymers, Stearyl Dimethicone Copolymers, Dimethicone Copolymers and mixtures there of.

The compositions may also include from about 0.1 to about 30 weight percent of natural fats or natural oils. More specifically, the compositions can include from about 0.5 to about 20 percent by weight of natural fats or natural oils. Desirably, the compositions include from about 1 to about 10 percent by weight of natural fats, natural oils or mixtures of both. Natural fats and oils include fats, oils, essential oils, fatty acids, fatty alcohols, phospholipids and mixtures of these compounds. The natural fats and oils can be similar to the lipids that are present in healthy skin in order to mimic the naturally present lipids. Synthetic or synthetically modified fats and oils could potentially also be used if they functioned in the same manner as their natural counterparts. Examples of fats and oils include Avocado Oil, Apricot Oil, Babassu Oil, Borage Oil, Camellia Oil, Canola Oil, Castor Oil, Coconut Oil, Corn Oil, Cottonseed Oil, Evening Primrose Oil, Hydrogenated Cottonseed Oil, Hydrogenated Palm Kernel Oil, Maleated Soybean Oil, Meadowfoam Oil, Palm Kernel Oil, Peanut Oil, Rapeseed Oil, Safflower Oil, Sphingolipids, Sweet Almond Oil, Tall Oil, Lanolin, Lanolin Alcohol, Lauric Acid, Palmitic Acid, Stearic Acid, Linoleic Acid, Stearyl Alcohol, Lauryl Alcohol, Myristyl Alcohol, Behenyl Alcohol, Rose Hip Oil, Calendula Oil, Chamomile Oil, Eucalyptus Oil, Juniper Oil, Sandlewood Oil, Tea Tree Oil, Sunflower Oil, Soybean Oil, PROLIPID 141 blend (available from International Specialty Products of Wayne, N.J.) and mixtures thereof.

The compositions may also include sterols, sterol derivatives or mixtures of both in an amount of from about 0.1 to about 10 percent by weight. Sterols and sterol derivatives include compounds such as .beta.-sterols with a tail on the 17 position and no polar groups, such as cholesterol, C.sub.10-C.sub.30 cholesterol/lanosterol esters, tall oil sterols, soy sterols, sterol esters and mixtures of these compounds. More specifically, the compositions include from about 0.5 to about 5 percent by weight of sterols, sterol derivatives or mixtures of both. Even more specifically, the compositions include from about 0.8 to about 3 percent by weight of the sterol compounds. Examples of suitable sterol compounds include cholesterol, sitosterol, stigmasterol, and ergosterol, as well as, C.sub.10-C.sub.30 cholesterol/lanosterol esters, cholecalciferol, cholesteryl hydroxystearate, cholesteryl isostearate, cholesteryl stearate, 7-dehydrocholesterol, dihydrocholesterol, dihydrocholesteryl octyidecanoate, dihydrolanosterol, dihydrolanosteryl octyidecanoate, ergocalciferol, tall oil sterol, soy sterol acetate, lanasterol, soy sterol, avocado sterols, “AVOCADIN” (trade name of Croda, Ltd. in Parsippany, N.J.), sterol esters and mixtures thereof.

The compositions further may include from about 0.1 to about 10 percent by weight of one or more emollients. More specifically, the compositions include from about 0.5 to about 5 percent by weight of emollient(s). Even more specifically, the compositions include from about 1 to about 5 percent by weight of emollient(s). Suitable emollients include petroleum based oils, petrolatum, vegetable oils, mineral oils, lanolin and its derivatives, fatty esters, glycerol esters and their derivatives, propylene glycol esters and their derivatives, alkoxylated carboxylic acids, alkyoxylated alcohols, fatty alcohols, alkyl methicones, alkyl dimethicones, phenyl silicones, alkyl trimethylsilanes, dimethicone and mixtures of such compounds.

The compositions can include the emollient and skin protectant, dimethicone. The dimethicone can be blended with the other components through the addition of water-based emulsions containing dimethicone such as emulsions having the trade designations “Dow Corning 1669 Emulsion” and “Dow Corning 1664 Emulsion” available from Dow Corning of Midland, Mich. The dimethicone can also be blended using a microencapsulated dimethicone such as are available from Lipo Technologies of Dayton, Ohio or from 3M of St. Paul, Minn. The dimethicone can also be added to the compositions of the invention in the form of an entrapped dimethicone. Dimethicone can be entrapped in “Polytrap” or “Microsponges” as are available from Advanced Polymer Systems of San Franciso, Calif. The dimethicone can also be incorporated in the form of a dimethicone treated powder such as dimethicone-treated talc or dimethicone-treated zinc oxide as are available from KOBO of South Plainfield, N.J.

Optionally, the compositions may include from about 1 percent by weight to about 20 percent by weight of one or more viscosity enhancers or rheology modifiers. The viscosity enhancers and rheology modifiers can be added to increase the melt point viscosity of the compositions. Increasing the melt point viscosity gives better stability of the compositions on the bodyfacing materials of the articles. The viscosity enhancers and rheology modifiers also improve the stability of the composition at the “hot box car” stability temperature of about 130.degree. F. (54.5.degree. C.). Suitable viscosity enhancers can include, but are not limited to, Actrylamides Copolymers, Agar, Gelatin, Water-Dispersable Metal Soaps, Butoxy Chitosan, Calcium Carboxymethyl Cellulose, Calcium Alginate, Carbomer, Carboxybutyl Chitosan, Carboxymethyl Chitosan, Carboxymethyl Dextran, Carboxymethyl Hydroxyethyl Cellulose, Cellulose Gum, DMAPA Acrylates/Acrylic Acid/Acrylonitrogens, Hectorite, Hydrated Silica, Hydroxyethyl Cellulose, Hydroxypropyl Guar, Hydroxypropyl Methylcellulose, Isobutylene/Sodium Maleate Copolymer, Kelp, Lithium Magnesium Silicate, Lithium Magnesium Sodium Silicate, Magnesium/Aluminum/Hydroxide/Carbonate, Magnesium Aluminum Silicate, Magnesium Silicate, Magnesium Trisilicate, Methoxy PEG-22/Dodecyl Glycol Copolymer, Metyl Cellulose, Metyl Hydroxyethylcellulose, Microcrystalline Cellulose, Montmorillonite, Nonoxynol Hydroxyethylcellulose, PEG Crosspolymer, Polyacrylate-3, Polyacrylic Acid, Polyethylene/isopropyl Maleate Copolymer, Polymethacrylic Acid, Polyvinyl Alcohol, PVP/Decene Copolymer, PVP Montmorillonite, Sodium Acrylates Copolymer, Sodium AcrylateNinyl Alcohol Copolymer, Sodium AcrylatesNinyl Isodecanate Crosspolymer, Sodium Carboxymethyl Starch, Sodium Hydroxypropyl Starch Phosphate, Sodium Polyacrylate, TEA Alginate, TEA Carbomer, Xanthan Gum, Yeast Polysaccharides and mixtures thereof.

In addition to the components already described, the compositions may also include active ingredients such as those ingredients that may be useful for treating skin irritations such as diaper rash. Examples of such active ingredients include allantoin and its derivatives, aluminum hydroxide gel, calamine, cocoa butter, cod liver oil, dimethicone, kaolin and its derivatives, lanolin and its derivatives, mineral oil, petrolatum, shark liver oil, talc, topical starch, zinc acetate, zinc carbonate, zinc oxide and mixtures of these ingredients. Some of the ingredients listed as possible active ingredients for treating the skin can also be used as emollients.

In order to enhance or increase the function of the lotion compositions, additional ingredients may be added. Examples of the classes of ingredients along with their functions include: antifoaming agents (reduce the tendency of foaming during processing); antimicrobial actives; antifungal actives; antiseptic actives; antioxidants (product integrity); antioxidants-cosmetic (reduce oxidation); astringents-cosmetic (induce a tightening or tingling sensation on skin); astringent-drug (a drug product that checks oozing, discharge or bleeding when applied to skin or mucous membrane and works by coagulating protein); biological additives (enhance the performance or consumer appeal of the product); colorants (impart color to the product); deodorants (reduce or eliminate unpleasant odor and protect against the formation of malodor on body surfaces); other emollients (help to maintain the soft, smooth and pliable appearance of the skin by their ability to remain on the skin surface or in the stratum corneum to act as lubricants, to reduce flaking and to improve the skin's appearance); external analgesics (a topically applied drug that has a topical analgesic, anesthetic or antipruritic effect by depressing cutaneous sensory receptors, or that has a topical counterirritant effect by stimulating cutaneous sensory receptors); film formers (to hold active ingredients on the skin by producing a continuous film on skin upon drying); fragrances (consumer appeal); silicones/organomodified silicones (protection, tissue water resistance, lubricity, tissue softness); oils (mineral, vegetable and animal); natural moisturizing agents (NMF) and other skin moisturizing ingredients known in the art; opacifiers (reduce the clarity or transparent appearance of the product); powders (enhance lubricity, oil adsorption, provide skin protection, astringency, opacity, etc.); skin conditioning agents; solvents (liquids employed to dissolve components found useful in the cosmetics or drugs); and surfactants (as cleansing agents, emulsifying agents, solubilizing agents and suspending agents).

Ranges are used to describe the relative quantities of compounds in the compositions and ranges are used to describe the relative physical properties of the compositions. It is understood that the ranges are by way of illustration only and that one of skill in the art would recognize that the nature of the specific compositions dictates the levels to be applied to achieve the desired results. The levels of components are ascertainable by routine experimentation in view of the present disclosure.

In addition to lotion compositions, the relatively low permeability areas 60 are also well suited to receiving other chemical compositions. For example, in an alternative embodiment, the relatively low permeability areas 60 are configured to receive an adhesive for adhering the body-side liner 42 to the absorbent structure 44. Of particular advantage, the relatively low permeability areas 60 may be configured so as to substantially prevent the adhesive composition from migrating through the liner and possibly irritating the skin of the user.

In general, any suitable adhesive composition may be applied to the liner for adhering the liner to the absorbent structure in accordance with the present invention. The adhesive composition may be, for instance, a spray adhesive or a hotmelt adhesive. The adhesive may be applied to the liner using any suitable application technique that is capable of placing the adhesive composition in the desired locations, such as slot coating.

The adhesive composition may contain, for instance, aliphatic polyolefins copolymers such as polyethylene-co-propylene copolymers, ethylene vinyl acetate copolymers, styrene-butadiene or styrene-isoprene block copolymers, a resin or analogous material such as a tackifier which may include hydrocarbons distilled from petroleum distillates, rosins and/or rosin esters, terpenes derived, for example, from wood or citrus, waxes, plasticizers or other materials to modify viscosity such as mineral oil, polybutene, paraffin oils, ester oils, and the like, and/or other additives including, but not limited to, antioxidants or other stabilizers.

In other embodiments, the adhesive composition may comprise a polyolefin, such as a polyethylene or a polypropylene. In still other embodiments, a starch adhesive or a modified starch adhesive may be used.

Examples of commercially available adhesives that may be used in the present invention include adhesive 5541 which is based on a styrene-butadiene-styrene block copolymer, adhesive NS-34-5610, adhesive 34-5610, adhesive 70-3998, and adhesive 33-2858, which are all available from The National Starch and Chemical Company of Bridgewater, N.J.

In one particular embodiment of the present invention, a lotion composition may be applied to one side of the liner 42 on the relatively low permeability areas 60, while on the opposite side of the liner an adhesive composition may be applied over the relatively low permeability areas 60 in order to adhere the liner to the absorbent structure 44. In this embodiment, the relatively low permeability areas 60 provide a placement location not only for a lotion composition but also for an adhesive composition.

The particular pattern that the relatively low permeability areas 60 form on the liner 42 may vary greatly depending upon the particular application, the chemical composition that is being topically applied to the liner, and the desired result. In FIG. 4, for instance, the relatively low permeability areas 60 form three spaced-apart columns. A greater or lesser number of columns may be provided. Further, in other embodiments, the relatively low permeability areas 60 may form a pattern that comprises discrete shapes or a reticulated pattern, such as a grid.

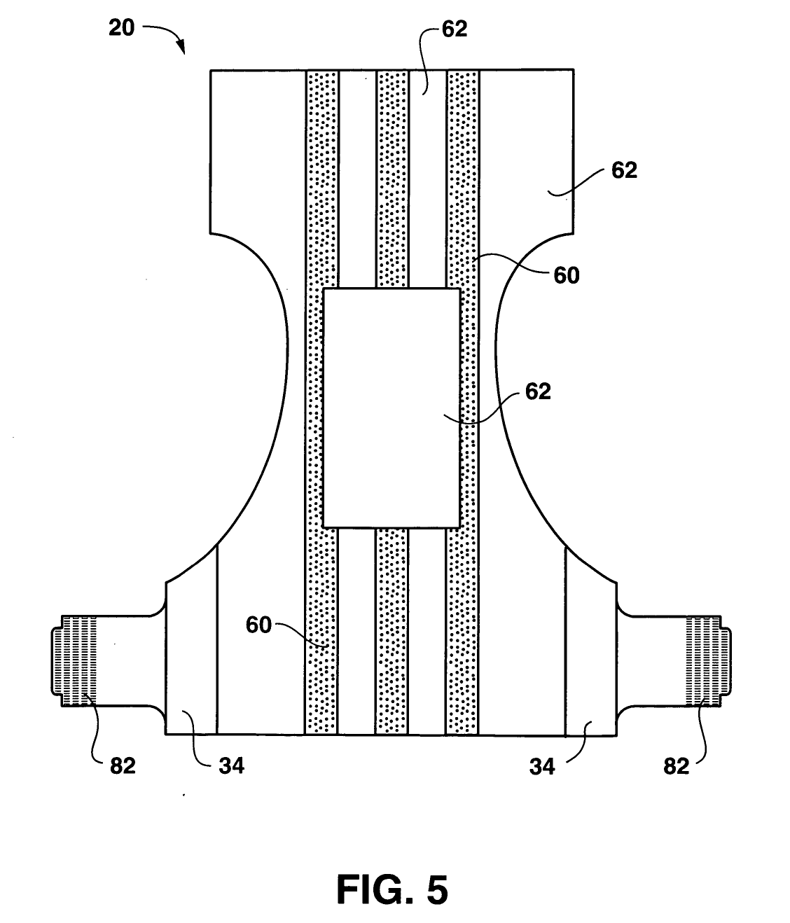

Referring to FIG. 5, an alternative embodiment of an absorbent structure 20 made in accordance with the present invention is shown. In the embodiment illustrated in FIG. 5, the relatively low permeability areas 60 are once again in the form of columns. In this embodiment, however, the columns are interrupted by an insult area comprising the relatively high permeability areas 62. The insult area is generally located in the crotch region and is provided for unimpeded liquid transfer during use of the article.

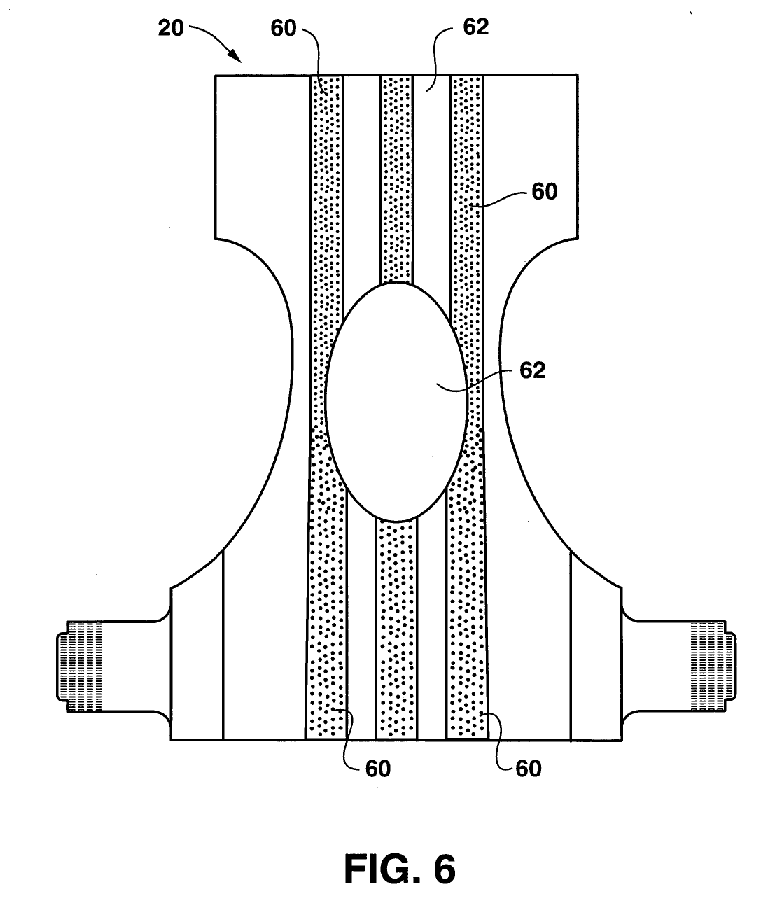

Referring to FIG. 6, another embodiment of an absorbent article 20 made in accordance with the present invention is shown. In this embodiment, once again, the relatively low permeability areas 60 are in the form of columns that extend from the back region to the front region of the article. The article further includes an insult area 62 that has a relatively high permeability for allowing rapid fluid transfer. In this embodiment, the insult area is in the shape of an oval as opposed to the rectangle shown in FIG. 5.

In FIG. 6, the columns of the relatively low permeability areas 60 are also wider in the back region of the article in comparison to the columns located on the front region. When applying a lotion composition to the article, for instance, a greater amount of the lotion composition may be desired in the back region where the skin is more prone to come into contact with fecal matter. Thus, in one embodiment, the lotion composition may cover a greater surface area in the back region.

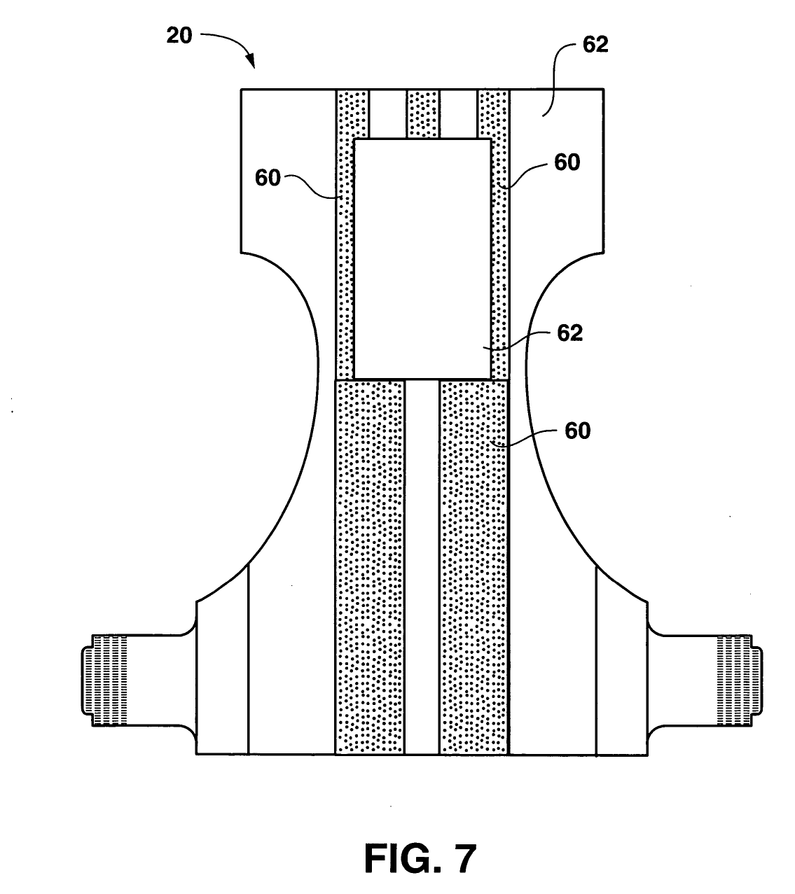

Referring to FIG. 7, still another embodiment of an absorbent article 20 made in accordance with the present invention is shown. In this embodiment, the insult area comprising the relatively high permeability area 62 is shifted forward, which may be more appropriate for a male wearer. In this embodiment, the relatively low permeability areas 60 comprise two wide columns in the back region and three narrower columns in the front region. Again, greater amounts of the lotion composition may be needed in the back region as opposed to the front region.

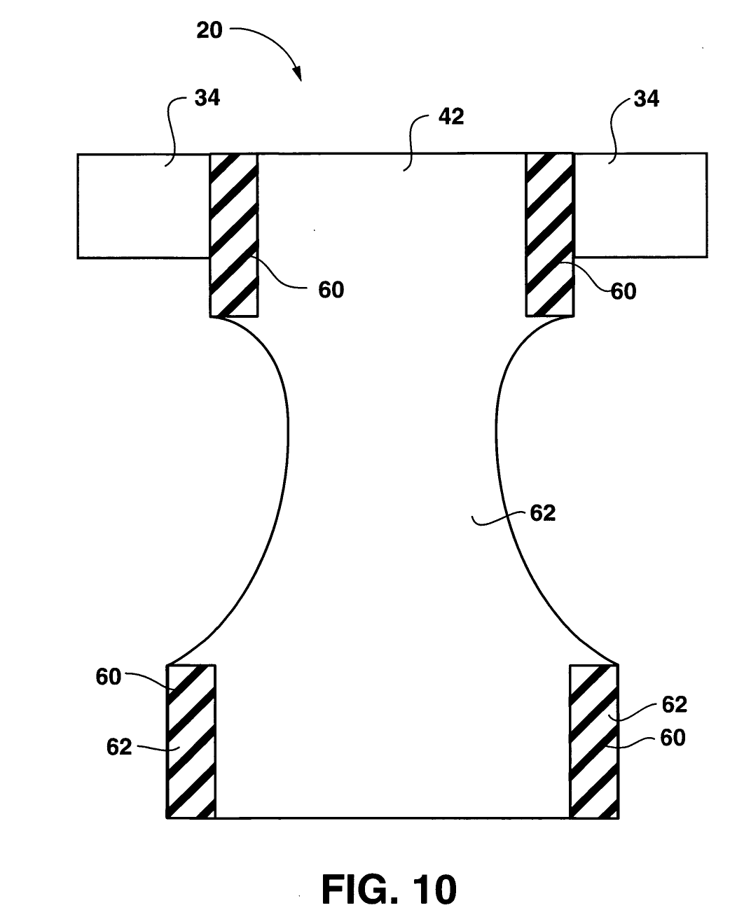

Referring to FIG. 10, another embodiment of an absorbent article 20 made in accordance with the present invention is shown. In this embodiment, the relatively low permeability areas 60 comprise diagonal lines that are only located at the corners of the article. The relatively low permeability areas 60 are separated from each other by relatively high permeability areas 62. Further, the majority of the surface area of the liner in the center of the article also comprises a relatively high permeability area. As illustrated, in this embodiment, relatively low permeability areas 60 only comprise a small fraction of the total surface area of the liner 42.

In the embodiment illustrated in FIG. 10, the relatively low permeability areas 60 are well suited for receiving an adhesive for adhering the liner 42 to an absorbent structure, to an outer cover, or to other components contained within the absorbent article.

In general, the lotion composition may be applied to the liner 42 so as to cover from about 5 percent to about 80 percent, and particularly from about 10 percent to about 70 percent of the surface area of the liner.

As described above, the absorbent article 20 includes the outer cover 40 and the absorbent structure 44. The outer cover 40 and the absorbent structure 44 may be made from many different materials depending upon the particular application and desired result. The layers, for instance, may be extendable and/or elastic.

The outer cover 40, for instance, may be breathable and/or may be liquid impermeable. The outer cover 40 may be constructed of a single layer, multiple layers, laminates, spunbond fabrics, films, meltblown fabrics, elastic netting, microporous webs, bonded card webs or foams provided by elastomeric or polymeric materials. The outer cover 40, for instance, can be a single layer of a liquid impermeable material, or alternatively can be a multi-layered laminate structure in which at least one of the layers is liquid impermeable. In other embodiments, however, it should be understood that the outer cover may be liquid permeable. In this embodiment, for instance, the absorbent article may contain an interior liquid barrier layer.

For instance, the outer cover 40 can include a liquid permeable outer layer and a liquid impermeable inner layer that are suitably joined together by a laminate adhesive, ultrasonic bonds, thermal bonds, or the like. Suitable laminate adhesives, which can be applied continuously or intermittently as beads, a spray, parallel swirls, or the like, can be obtained from Bostik Findley Adhesives, Inc., of Wauwatosa, Wis., U.S.A., or from National Starch and Chemical Company, Bridgewater, N.J. U.S.A. The liquid permeable outer layer can be any suitable material and is desirably one that provides a generally cloth-like texture. One example of such a material is a 20 gsm (grams per square meter) spunbond polypropylene nonwoven web. The outer layer may also be made of those materials of which the liquid permeable bodyside liner 42 is made.

The inner layer of the outer cover 40 can be both liquid and vapor impermeable, or it may be liquid impermeable and vapor permeable. The inner layer can be manufactured from a thin plastic film, although other flexible liquid impermeable materials may also be used. The inner layer, or the liquid impermeable outer cover 40 when a single layer, prevents waste material from wetting articles, such as bed sheets and clothing, as well as the wearer and caregiver. A suitable liquid impermeable film for use as a liquid impermeable inner layer, or a single layer liquid impermeable outer cover 40, is a 0.02 millimeter polyethylene film commercially available from Pliant Corporation of Schaumburg, Ill., U.S.A.

The outer cover 40 may be extendable and optionally elastic. Elastic non-woven laminate webs that can be used as the outer cover 40 include a non-woven material joined to one or more gatherable non-woven webs, films, or foams. Stretch Bonded Laminates (SBL) and Neck Bonded Laminates (NBL) are examples of elastomeric composites. Non-woven fabrics are any web of material which has been formed without the use of textile weaving processes which produce a structure of individual fibers that are interconnected in an integrating manner.

Examples of suitable materials are spunbond-meltblown fabrics, spunbond-meltblown-spunbond fabrics, spunbond fabrics, or laminates of such fabrics with films, foams, or other nonwoven webs. Elastomeric materials may include cast or blown films, foams, meltblown fabrics or spunbond fabrics composed of polyethylene, polypropylene, or polyolefin elastomers, as well as combinations thereof. The elastomeric materials may include PEBAX elastomer (available from AtoChem located in Philadelphia, Pa.), HYTREL elastomeric polyester (available from Invista of Wilmington, Del.), KRATON elastomer (available from Kraton Polymers of Houston, Tex.), or strands of LYCRA elastomer (available from Invista of Wilmington, Del.), or the like, as well as combinations thereof. The outer cover 40 may include materials that have elastomeric properties through a mechanical process, printing process, heating process, or chemical treatment. For examples such materials may be apertured, creped, neck-stretched, heat activated, embossed, and micro-strained; and may be in the form of films, webs, and laminates.

Alternatively, the outer cover 40 may include a woven or non-woven fibrous web layer which has been totally or partially constructed or treated to impart the desired levels of liquid impermeability to selected regions that are adjacent or proximate the absorbent structure. For example, the outer cover 40 may include a gas-permeable, non-woven fabric layer laminated to a polymer film layer which may or may not be gas-permeable. Other examples of fibrous, cloth-like outer cover 40 materials can include a stretch thinned or stretch thermal laminate material composed of a 0.6 mil (0.015 mm) thick polypropylene blown film and a 0.7 osy (23.8 gsm) polypropylene spunbond material (2 denier fibers).

The absorbent structure 44, on the other hand, can be any structure or combination of components which are generally compressible, conformable, non-irritating to a wearer's skin, and capable of absorbing and retaining liquids and certain body wastes. For example, the absorbent structure 44 may include an absorbent web material of cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof. In a particular aspect, the absorbent web material is a matrix of cellulosic fluff and superabsorbent hydrogel-forming particles. The cellulosic fluff may include a blend of wood pulp fluff. One preferred type of fluff is identified with the trade designation CR 1654, available from Bowater of Greenville, S.C., USA, and is a bleached, highly absorbent sulfate wood pulp containing primarily southern soft wood fibers. The absorbent materials may be formed into a web structure by employing various conventional methods and techniques. For example, the absorbent web may be formed with a dry-forming technique, an air forming technique, a wet-forming technique, a foam-forming technique, or the like, as well as combinations thereof. Methods and apparatus for carrying out such techniques are well known in the art. Furthermore, the absorbent structure may itself encompass multiple layers in the Z direction. Such multiple layers may take advantage of differences in absorbency capacity, such as by placing a lower capacity absorbent material layer closer to the liner 42 and a higher capacity absorbent material closer to the outer cover layer 40. Likewise, discrete portions of an absorbent single-layered structure may encompass higher capacity absorbents, and other discrete portions of the structure may encompass lower capacity absorbents.

As a general rule, the superabsorbent material is present in the absorbent web in an amount of from about 0 to about 90 weight percent based on total weight of the web. The web may have a density within the range of about 0.10 to about 0.60 grams per cubic centimeter.

Superabsorbent materials are well known in the art and can be selected from natural, synthetic, and modified natural polymers and materials. The superabsorbent materials can be inorganic materials, such as silica gels, or organic compounds, such as crosslinked polymers. Typically, a superabsorbent material is capable of absorbing at least about 10 times its weight in liquid, and desirably is capable of absorbing more than about 25 times its weight in liquid. Suitable superabsorbent materials are readily available from various suppliers. For example, SXM 9394, and Favor 9543 superabsorbents are available from DeGussa Superabsorbers.

After being formed or cut into a desired shape, the absorbent web material may be wrapped or encompassed by a suitable tissue or meltblown web or the like wrap sheet that aids in maintaining the integrity and shape of the absorbent structure 44.

The absorbent web material may also be a coform material. The term “coform material” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles, inorganic absorbent materials, treated polymeric staple fibers and the like. Any of a variety of synthetic polymers may be utilized as the melt-spun component of the coform material. For instance, in certain aspects, thermoplastic polymers can be utilized. Some examples of suitable thermoplastics that can be utilized include polyolefins, such as polyethylene, polypropylene, polybutylene and the like; polyamides; and polyesters. In one aspect, the thermoplastic polymer is polypropylene. Some examples of such coform materials are disclosed in U.S. Pat. No. 4,100,324 to Anderson, et al.; U.S. Pat. No. 5,284,703 to Everhart, et al.; and U.S. Pat. No. 5,350,624 to Georger, et al.; which are incorporated herein by reference to the extent they are consistent (i.e., not in conflict) herewith.

In a particular aspect of the absorbent article of the present invention, the absorbent structure 44 may also be elastomeric. For this purpose, the absorbent web material can include elastomeric fibers in an amount which is at least a minimum of about 2 wt %. The amount of elastomeric fibers can alternatively be at least about 3 wt %, and can optionally be at least about 5 wt % to provide improved performance. In addition, the amount of elastomeric fibers can be not more than about 60 wt %. Alternatively, the amount of elastomeric fibers can be not more than about 45 wt %, and optionally, can be not more than about 30 wt % to provide improved benefits. These values may impact the absorbent structure 44 by affecting the desired levels of stretchability and structural stability without excessively degrading the physical properties or the liquid-management properties of the absorbent structure. An absorbent web material with an excessively low proportion of elastomeric fibers may be insufficiently stretchable, and a web material with an excessively high proportion of elastomeric fibers may exhibit an excessive degradation of its absorbency functionalities, such as poor intake, poor distribution, poor retention of liquid.

The absorbent structure 44 may include an elastomeric coform absorbent web material. Such materials are described for instance in U.S. Pat. Nos. 6,231,557 B1 and 6,362,389 B1, which are each incorporated by reference herein to the extent they are consistent (i.e., not in conflict) herewith.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. An absorbent article comprising:
an absorbent structure;
a body-side liner having a first side and a second side, the first side being located adjacent to the absorbent structure, the second side of the body-side liner being configured to be placed next to a wearer of the absorbent article, the liner including at least one area of relatively high permeability and at least one area of relatively low permeability, the area of high permeability being configured to allow liquids to flow therethrough for absorption by the absorbent structure; and
a lotion composition applied to the second side of the liner, the lotion composition being located on the relatively low permeability area of the liner, the relatively low permeability area inhibiting the lotion composition from migrating through the liner to the absorbent structure.

2. An absorbent article as defined in claim 1, further comprising an outer cover, the absorbent structure being positioned between the liner and the outer cover.

3. An absorbent article as defined in claim 1, wherein the liner includes a plurality of the relatively low permeability areas arranged in a pattern.

4. An absorbent article as defined in claim 1, wherein the lotion composition is applied only to the at least one relatively low permeability area.

5. An absorbent article as defined in claim 3, wherein the pattern of relatively low permeability areas comprises columns.

6. An absorbent article as defined in claim 5, wherein the absorbent article includes a front region, a back region, and a crotch region positioned in between the front region and the back region, the columns extending generally from the front region to the back region.

7. An absorbent article as defined in claim 6, wherein the pattern of columns is interrupted by an insult zone comprising the relatively high permeability area, the insult zone positioned generally in the crotch region.

8. An absorbent article as defined in claim 1, wherein the lotion composition comprises a material selected from the group consisting of a hydrophilic solvent, a high molecular weight polyethylene glycol, a fatty alcohol, an emulsifying surfactant, a natural fat, a natural oil, a sterol, a sterol derivative, an emollient, and mixtures thereof.

9. An absorbent article as defined in claim 1, wherein the bodyside liner comprises a nonwoven material and the relatively low permeability area is formed by applying a film-forming material to the nonwoven material.

10. An absorbent article as defined in claim 9, wherein the film-forming material is water soluble.

11. An absorbent article as defined in claim 9, wherein the film-forming material comprises a material selected from the group consisting of polyvinyl alcohol, polyvinyl acetate, polyethylene oxide, polypropylene oxide, polyacrylic acid salts, polyacrylamide, cellulose ethers, modified collagen gelatins, alginates, and modified starches.

12. An absorbent article as defined in claim 1, wherein the body-side liner comprises a nonwoven material containing synthetic fibers, and wherein the relatively low permeability areas are thermoformed or pressure formed into the nonwoven material.

13. An absorbent article as defined in claim 1, wherein the relatively low permeability area comprises a film.

14. An absorbent article as defined in claim 1, wherein the relatively low permeability area comprises a laminate.

15. An absorbent article as defined in claim 1, wherein the body-side liner comprises a nonwoven web, the relatively high permeability area being made from the same material as the relatively low permeability area, the relatively low permeability area having a higher basis weight than the relatively high permeability area.

16. An absorbent article as defined in claim 1, further comprising an adhesive composition applied to the first side of the liner, the adhesive composition being located on the relatively low permeability area of the liner, the adhesive composition joining the liner to the absorbent structure, the relatively low permeability area inhibiting the adhesive composition from migrating through to the second side of the liner.

17. An absorbent article as defined in claim 16, wherein the adhesive composition comprises a hotmelt adhesive.

18. An absorbent article as defined in claim 16, wherein the adhesive composition comprises a spray adhesive.

19. An absorbent article as defined in claim 16, wherein the liner includes a pattern of relatively low permeability areas, the adhesive composition being applied only to the relatively low permeability areas.

20. An absorbent article as defined in claim 16, wherein the adhesive composition comprises a styrene-butadiene copolymer, a styrene-butadiene-styrene copolymer, a styrene-isoprene copolymer, an aliphatic polyolefin copolymer, an ethylene vinyl acetate copolymer, or a starch-based adhesive.

21. An absorbent article comprising:
an absorbent structure:
a body-side liner having a first side and a second side, the first side being located adjacent to the absorbent structure, the second side of the body-side liner being configured to be placed next to a wearer of the absorbent article, the liner including at least one area of relatively high permeability and at least one area of relatively low permeability, the area of high permeability being configured to allow liquids to flow therethrough for absorption by the absorbent structure; and
an adhesive composition applied to the first side of the liner, the adhesive composition being located on the relatively low permeability area of the liner, the relatively low permeability area inhibiting the adhesive composition from migrating through to the second side of the liner, the adhesive composition joining the liner to the absorbent structure.

22. An absorbent article as defined in claim 21, further comprising an outer cover, the absorbent structure being positioned between the liner and the outer cover.

23. An absorbent article as defined in claim 21, wherein the liner includes a plurality of the relatively low permeability areas arranged in a pattern.

24. An absorbent article as defined in claim 21, wherein the adhesive composition is applied only to the at least one relatively low permeability area.

25. An absorbent article as defined in claim 23, wherein the pattern of relatively low permeability areas comprises columns.

26. An absorbent article as defined in claim 25, wherein the absorbent article includes a front region, a back region, and a crotch region positioned in between the front region and the back region, the columns extending generally from the front region to the back region.

27. An absorbent article as defined in claim 26, wherein the pattern of columns is interrupted by an insult zone comprising the relatively high permeability area, the insult zone positioned generally in the crotch region.

28. An absorbent article as defined in claim 21, wherein the bodyside liner comprises a nonwoven material and the relatively low permeability area is formed by applying a film-forming material to the nonwoven material.

29. An absorbent article as defined in claim 21, wherein the body-side liner comprises a nonwoven material containing synthetic fibers, and wherein the relatively low permeability areas are thermoformed into the nonwoven material.

30. An absorbent article as defined in claim 21, wherein the relatively low permeability area comprises a film.

31. An absorbent article as defined in claim 21, wherein the relatively low permeability area comprises a laminate.

32. An absorbent article as defined in claim 21, wherein the body-side liner comprises a nonwoven web, the relatively high permeability area being made from the same material as the relatively low permeability area, the relatively low permeability area having a higher basis weight than the relatively high permeability area.

33. An absorbent article as defined in claim 21, wherein the adhesive composition comprises a hotmelt adhesive.

34. An absorbent article as defined in claim 21, wherein the adhesive composition comprises a spray adhesive.

35. An absorbent article as defined in claim 21, wherein the adhesive composition comprises a styrene-butadiene copolymer, a styrene-butadiene-styrene copolymer, a styrene-isoprene copolymer, an aliphatic polyolefin copolymer, an ethylene vinyl acetate copolymer, or a starch-based adhesive.

36. An absorbent article as defined in claim 21, further comprising a lotion composition applied to the second side of the body-side liner, the lotion composition being located on the relatively low permeability area of the liner, the relatively low permeability area inhibiting the lotion composition from migrating through the liner to the absorbent structure.

37. An absorbent article as defined in claim 36, wherein the lotion composition comprises a material selected from the group consisting of a hydrophilic solvent, a high molecular weight polyethylene glycol, a fatty alcohol, an emulsifying surfactant, a natural fat, a natural oil, a sterol, a sterol derivative, an emollient, and mixtures thereof.

38. An absorbent article as defined in claim 36, wherein the liner includes a pattern of relatively low permeability areas, the lotion composition being applied only to the relatively low permeability areas.

39. An absorbent article comprising:
an outer cover;
an absorbent structure;
a body-side liner having a first side and a second side, the absorbent structure being positioned in between the outer cover and the body-side liner, the first side of the body-side liner being located adjacent to the absorbent structure, the second side of the body-side liner being configured to be placed next to a wearer of the absorbent article, the liner including a pattern of relatively high permeability areas and relatively low permeability areas, the areas of high permeability being configured to allow liquids to flow therethrough for absorption by the absorbent structure;
an adhesive composition applied to the first side of the liner for joining the liner to the absorbent structure, the adhesive composition being located only at the relatively low permeability areas of the liner, the relatively low permeability areas inhibiting the adhesive composition from migrating through the liner to the second side; and
a lotion composition applied to the second side of the body-side liner, the lotion composition being located only on the relatively low permeability areas, the relatively low permeability areas inhibiting the lotion composition from migrating through the liner to the absorbent structure.

40. An absorbent article as defined in claim 39, wherein the bodyside liner comprises a nonwoven material and the relatively low permeability area is formed by applying a film-forming material to the nonwoven material.

41. An absorbent article as defined in claim 39, wherein the body-side liner comprises a nonwoven material containing synthetic fibers, and wherein the relatively low permeability areas are thermoformed into the nonwoven material.

42. An absorbent article as defined in claim 39, further comprising an adhesive composition applied to the first side of the liner, the adhesive composition being located on the relatively low permeability area of the liner, the adhesive composition joining the liner to the absorbent structure, the relatively low permeability area inhibiting the adhesive composition from migrating through to the second side of the liner, and wherein the lotion composition comprises a material selected from the group consisting of a hydrophilic solvent, a high molecular weight polyethylene glycol, a fatty alcohol, an emulsifying surfactant, a natural fat, a natural oil, a sterol, a sterol derivative, an emollient, and mixtures thereof.
